(19) United States
(12) Patent Application Publication
(10) Pub. No.: US 2026/0207925 A1
(43) Pub. Date: Jul. 23, 2026

(54) STIMULATION ELECTRODE ARRANGEMENT FOR A DEVICE FOR ELECTROSTIMULATION OF THE EYE, DEVICE AND SYSTEM FOR ELECTROSTIMULATION OF THE EYE, SET WITH A STIMULATION ELECTRODE ARRANGEMENT, AND METHOD

(71) Applicant: Okuvision GmbH, Reutlingen (DE)

(72) Inventors: Nicolò Ceretto Gianone, Torino (IT); Paul Henzler, Nuertingen (DE)

(73) Assignee: Okuvision GmbH, Reutlingen (DE)

(21) Appl. No.: 19/569,852

(22) Filed: Mar. 17, 2026

Related U.S. Application Data
(63) Continuation of application No. PCT/EP2024/075869, filed on Sep. 17, 2024.

(30) Foreign Application Priority Data
Sep. 18, 2023 (DE) 10 2023 125 137.6

(51) Int. Cl. A61N 1/04

(52) U.S. Cl. CPC A61N 1/0408; A61N 1/0472

(57) ABSTRACT

The present disclosure relates to a stimulation electrode arrangement (20) for a device (10) for electrostimulation of the eye, wherein the device (10) comprises a spectacle-like frame (11) with a nose part (12), an arrangement connected to the nose part (12) for holding the frame on the head of the patient, and at least one electrode holder (30) arranged at the frame (11) for a stimulation electrode (21), in particular a wire-shaped stimulation electrode, wherein the stimulation electrode arrangement (20) comprises a first electrode carrier (22) and a second electrode carrier (23), wherein the stimulation electrode (21) is arranged between the first electrode carrier (22) and the second electrode carrier (23), wherein the first and/or second electrode carrier is adapted to be magnetically attached to the electrode holder (30).

### CROSS-REFERENCE TO RELATED APPLICATION

This application is a continuation of International Patent Application No. PCT/EP2024/075869, filed on Sep. 17, 2024, which claims priorty to German Patent Application No. 10 2023 125 137.6, filed on Sep. 18, 2023, the disclosures of which are hereby incorporated herein by reference in their entireties.

### TECHNICAL FIELD

The present disclosure relates to a stimulation electrode arrangement for a device for electrostimulation of the eye, in particular for transcorneal electrostimulation, wherein the device has a spectacle-like frame with a nose part, an arrangement connected to the nose part for holding the frame on the head of the patient, and at least one electrode holder arranged at the frame for a stimulation electrode arrangement.

The present disclosure further relates to a corresponding device for electrostimulation of the eye, a system with a stimulation electrode arrangement and corresponding device, a set with a stimulation electrode arrangement, and a method for attaching a stimulation electrode arrangement to a device for electrostimulation of the eye.

### BACKGROUND

WO 2011/086150 A2 generally discloses stimulation electrodes, electrode holders, and devices for electrostimulation.

The known stimulation electrode is a wire-shaped electrode which is attached to the spectacle-like frame. When the frame is being arranged on the head of a patient, the flexible wire electrode comes during putting on the spectacle-like frame into contact with the front surface of the eye in the lower edge region of the cornea of the eye. A counter electrode is either provided on the frame or is attached separately at the patient, for example on forehead of the head or on an earlobe.

The wire electrode and the counter electrode can be used to route a pulse-like electrical stimulation signal into the eye, leading to a stimulation current flowing via the cornea into the eye.

The known device and other devices are used to route electrical stimulation signals into the eye because it was found that this can stabilize or even improve certain forms of retinopathia pigmentosa and other eye diseases.

Retinal degeneration is a significant cause of blindness in industrialized countries. Various trials provide indications that low electrical currents which flow through the retina can delay the onset of genetic, age-related or sudden degeneration of the retina.

This opens up the possibility of being able to delay the gradual loss of sight through regular, that is to say e.g. daily or weekly, electrical stimulation of the eye in order thus to maintain parts of the vision for longer in affected patients, or even restore parts of the vision.

Expediently, this treatment should be carried out by the patient himself and at home. In doing so, in the extreme case, the assumption must be made that the patient is practically blind. The result of this is that there are significant demands on the device and the stimulation electrode mentioned at the outset.

This is because the wire-shaped stimulation electrode must be replaced after every use, or at least very frequently. On the one hand, the wire-shaped stimulation electrode is a very thin electrode, which can break in the case of improper or even frequent use.

On the other hand, considerations in respect of hygiene may make it necessary to replace the stimulation electrode after each use.

WO 2011/086150 A2, mentioned at the outset, describes three different embodiments for these applications.

In a first embodiment, the device for electrostimulation of the eye is designed in the style of spectacles, with spiral electrode holders, which extend towards the eye, thereby being provided on the frame which is usually provided for the lenses. Two such electrode holders are provided for each eye, and so a wire electrode needs to be connected to the two inner ends of these flexible electrode holders in order to come into contact with the cornea when the spectacles are put on.

In a second embodiment, a facemask is shown, which is fitted over an area to parts of the face of the patient. Eye openings, on which pushbuttons are provided, into which the wire-shaped stimulation electrodes are hooked, are provided in this facemask.

In a third embodiment, a type of “shooting spectacles” is shown, in which the spectacles-like frame comprises a nose clip and two eye brackets coming off the former, which eye brackets are in turn connected to foldable earpieces.

Each two electrode holders are attached to the eye brackets; said electrode holders can be adjusted along the eye bracket and perpendicular to the eye bracket, i.e., in all three directions.

On their inner free ends, the electrode holders are respectively connected to ears, into which the wire-shaped stimulation electrode is introduced. The stimulation electrode is attached with its first end to the nose-side electrode holder and is then routed through an ear to the temple-side electrode holder. At its second end, the stimulation electrode is weighed down by a weight such that the stimulation electrode is pretensioned in this manner.

A person skilled in the art fits this frame to the head of the patient such that, when such spectacles which are equipped with one or two stimulation electrodes are put on, the stimulation electrode automatically rests against the eye.

While a person skilled in the art sets the geometric conditions of the frame on one occasion, the patient should subsequently use the device at home in order to subject his eyes to regular electrical stimulation.

However, hooking the stimulation electrode into the ears or onto the electrode holder, which is required for this, is found to be extremely difficult for patients with visual impairments. In part, the thin electrode thread is not recognized by the patients or said electrode thread can slide out of the electrode holders when the spectacles frame is put on.

In order to prevent this, the stimulation electrode must be wound a number of times around the holder at the nose-side electrode holder. This requires much dexterity because, firstly, the thread is rigid and, secondly, the patients have to resort to their sense of touch as a result of their visual impairment.

Against this background, DE 10 2011 055 844 B4 discloses a further improved stimulation electrode arrangement for a device for electrostimulation of the eye. The device comprises a spectacle-like frame with a nose part, an arrangement connected to the nose part for holding the frame on the head of the patient, and at least one electrode holder, which is arranged on the nose part in adjustable fashion, for the wire-shaped stimulation electrode. The stimulation electrode arrangement comprises an electrode carrier, which can be attached to the electrode holder in detachable fashion and on which the stimulation electrode is arranged. In an embodiment, the electrode carrier is formed as a U-shaped bracket with two arms and a groove open to the outside. The solution described in DE 10 2011 055 844 B4 provides a stimulation electrode arrangement of the type mentioned above, which is easy to handle and also facilitates handling for visually impaired patients.

In the solution described in DE 10 2011 055 844 B4, the patient shall only need to grip the stimulation electrode arrangement by the electrode carrier, which is much more massive than the thin stimulation electrode, and attach this electrode carrier in the electrode holder which is already present and adjusted on the device.

Since the electrode carrier has a greater mass and greater dimensions than the stimulation electrode itself, visually impaired or even blind patients can feel the electrode carrier and insert it, in the correct position, into the electrode holder.

### SUMMARY

In view of the above, it is an object of aspects of the present disclosure to further improve a stimulation electrode arrangement of the type mentioned at the outset such that that handling for visually impaired users is further improved. Furthermore, it would be desirable to provide a stimulation electrode arrangement of the type mentioned at the outset which can be flexibly applied by different users.

According to a first aspect of the present disclosure, it is therefore proposed to provide a stimulation electrode arrangement for a device for electrostimulation of the eye, wherein the device comprises a spectacle-like frame with a nose part, an arrangement connected to the nose part for holding the frame on the head of the patient, and at least one electrode holder (adjustably) arranged at the frame, in particular at the nose part, for a (wire-shaped) stimulation electrode or for a stimulation electrode arrangement with a (wire-shaped) stimulation electrode, wherein the stimulation electrode arrangement comprises a first electrode carrier and a second electrode carrier, wherein the stimulation electrode is arranged between the first electrode carrier and the second electrode carrier, wherein the first and/or second electrode carrier is adapted to be magnetically attached to the electrode holder.

The solutions described in the earlier applications mentioned at the outset already significantly improve the handling of a stimulation electrode for electrostimulation of the eye for visually impaired users. However, the precise placement of the stimulation electrode arrangement on the electrode holder may continue to cause difficulties for visually impaired users.

It is therefore proposed that the stimulation electrode arrangement comprises a first electrode carrier and a second electrode carrier, wherein the stimulation electrode is arranged between the first electrode carrier and the second electrode carrier, wherein the first and/or second electrode carrier is adapted to be magnetically attached to the electrode holder. By magnetically attaching the electrode carrier to the electrode holder, handling is further facilitated for visually impaired users. The electrode carrier and electrode holder attract each other magnetically, allowing a certain degree of self-positioning of the electrode carrier and electrode holder relative to each other. Approximate or imperfect positioning of the electrode holder, as it can also be achieved by a visually impaired user, can therefore be sufficient to magnetically receive the electrode carrier.

A further advantage of the proposed solution can be that hygiene can be improved when handling a stimulation electrode arrangement for a device for electrostimulation of the eye. Due to the electrode carrier adapted to be magnetically attached to the electrode holder, fewer attempts by the visually impaired user to correctly attach the electrode carrier to the electrode holder may be required. This reduces the risk of contamination being inserted into the eye due to improper handling and further improves hygiene during handling.

A further advantage can be that the proposed stimulation electrode arrangement can be flexibly used by different users. The solution described in WO 2013079357 A1 and the granted German patent DE102011055844B4 from this patent family proposes a single electrode holder with a U-shaped bracket. However, such a bracket would have to be adapted to the user as needed, or different variations would have to be provided for different users. This increases the number of variants and leads to additional inventory effort.

Aspects of the present disclosure, on the other hand, propose providing a first and a second electrode carrier, thus in particular, two separate electrode carriers, between which the stimulation electrode is arranged. An advantage can be that the two electrode carriers can be separately attached to one or more electrode holders. The electrode holders can be pre-adjusted on the frame, in particular on the nose part, of the device for electrostimulation of the eye. Preferably, apart from the (flexible) stimulation electrode, there is no mechanical connection between the first and second electrode carriers, in particular no bracket with a fixed geometry. The electrode carriers are simply applied to these pre-adjusted electrode holders. A user-specific adjustment of the stimulation electrode arrangement, i.e., adjustment of the element to be replaced each use, is hence no longer necessary. This further improves handling, in particular for visually impaired users. Furthermore, the number of variants and the effort required for inventory can be reduced.

A further advantage can be that the stimulation electrode and the first and second electrode carriers form an economical unit as a stimulation electrode arrangement, which can be provided pre-assembled as a disposable item.

In the context of the present disclosure, a stimulation electrode arrangement can refer to an electrode assembly for stimulating and/or measuring. Accordingly, a stimulation electrode can refer to an electrode for stimulating and/or measuring. In other words, not only can an electrical stimulation signal be delivered, but a measurement signal can also be recorded. For example, with the stimulation electrode or stimulation electrode arrangement, a stimulus can be applied or an electroretinogram or ERG signal can be acquired. Accordingly, in the context of the present disclosure, a device for electrostimulation of the eye can refer to a device for stimulating and/or measuring. Accordingly, in the context of the present disclosure, a system for electrostimulation of the eye can refer to a system for stimulating and/or measuring.

Thereby, it is advantageous if the first electrode carrier is adapted to be magnetically attached to the at least one electrode holder and the second electrode carrier is adapted to be magnetically attached to the at least one electrode holder. The first and second electrode carriers can be attached to a common electrode holder. However, two electrode holders can also be provided. The first electrode carrier can be adapted to be magnetically attached to a first electrode holder. The second electrode carrier can be adapted to be magnetically attached to a second electrode holder. An advantage of this embodiment can be that the first and second electrode holders can each be adjusted as desired to user. In particular, the first and second electrode holders can be adapted to be adjusted separately from each other.

The first electrode carrier can be a nasal electrode carrier and can be adapted to be magnetically attached to a nasal receptacle of the electrode holder. The second electrode carrier can be a temporal electrode carrier and can be adapted to be magnetically attached to a temporal receptacle of the electrode holder. Hereby, it is also possible that two electrode holders are provided, a first, nasal electrode holder and a second temporal electrode holder. The nasal electrode holder can also be referred to as a nose-side electrode holder. The temporal electrode holder can also be referred to as a temple-side electrode holder. The receptacle can, for example, be a receptacle pin onto which the electrode carrier, e.g., a funnel-shaped electrode carrier, is placed.

In an embodiment, the first and/or second electrode carrier can comprise a magnet and can be adapted to be attached to the electrode holder by the magnet. Alternatively or in addition, the at least one electrode holder can comprise a magnet and the electrode carrier can be adapted to be attached to the electrode holder by the magnet. For example, two electrode holders can be provided, each having a magnet. An advantage of arranging the magnet or magnets on the electrode holder can be that the magnets can be reused repeatedly for multiple applications. It may therefore be sufficient to provide no magnets in the electrode carriers of the stimulation electrode arrangement, but only a (magnetizable) counterpart which is attracted by the magnet in the electrode holder. This can reduce the costs and material usage for the stimulation electrode arrangement. This is particularly advantageous because hygienic considerations may require the stimulation electrode to be replaced regularly, in particular after every use.

The first and/or second electrode carrier can be adapted to be attached self-aligning at the electrode holder. The electrode holder and electrode carrier are thus magnetically attached to each other and also orient themselves at each other. For example, the electrode holder and electrode carrier can be configured to be self-centering. An advantage of this embodiment can be that handling for a visually impaired user is further improved.

In a refinement, the first and/or second electrode carrier can comprise a funnel and can be adapted to be attached in a self-aligning manner at the electrode holder by the funnel. Alternatively, the first and/or second electrode holder can comprise a funnel, and the electrode carrier and electrode holder can be adapted to be attached to each other in a self-aligning manner by the funnel. An advantage of this embodiment can be that handling for a visually impaired user is further improved. A tapered funnel shape allows the visually impaired user to roughly position the electrode carrier and electrode holder in the funnel opening and to position the elements correctly by the guidance provided by the funnel. A funnel can be refer to as a “little cup” or a receptacle with a tapered shape. The funnel can further be adapted to prevent tilting or tipping of the electrodes and electrode holder.

In a refinement, an electrical contact can be arranged at a bottom of the funnel, which is electrically conductively connected with the stimulation electrode. An advantage of this embodiment can be that an electrical contact with the stimulation electrode can be established when attaching the funnel.

In a refinement, a magnetizable metal element can be arranged at the bottom of the funnel. The magnetizable metal element can be adapted to provide both an electrical contact to the stimulation electrode and to be magnetically attached to the electrode holder. The magnetizable metal can provide an electrical contact which is electrically conductively connected to the stimulation electrode. A synergy effect can thus be achieved in that the attachment and current transmission of the stimulation electrode arrangement can be provided with the same element.

The first and/or second electrode carrier can be adapted to be attached in a defined orientation at the at least one electrode holder, in particular wherein the at least one electrode holder can comprise an oval receptacle for the first and/or second electrode carrier. For example, a first and a second electrode holder can be provided, each having an oval receptacle for the first and second electrode carriers. The first and second electrode carriers can, for example, comprise an oval funnel corresponding to the oval receptacle, which can be attached to it in a self-centering and self-aligning manner. As a receptacle, the electrode holder can, for example, comprise a receptacle pin. The funnel of the electrode carrier can be slipped onto such a receptacle pin. This facilitates handling for a visually impaired user. In the context of the present disclosure, oval can refer to longer in a first direction than in a second direction orthogonal thereto.

The stimulation electrode can be a so-called DTL electrode. The stimulation electrode can be made of a wire made of a noble metal or a stainless steel alloy or a metal-coated plastic thread. The DTL electrode can be a metal-coated plastic thread, in particular a silver-coated nylon thread.

The stimulation electrode can be or comprise a contact lens provided with at least one wire-shaped connection with electrode structures, in particular with electrode structures made of a noble metal. An advantage of this embodiment can be a more areal stimulation and improved input of the stimulation signal.

The stimulation electrode can at least at its first end be electrically conductively connected to the first electrode carrier. The electrode carrier can be adapted to be electrically conductively connected to the electrode holder. With this measure, it can be advantageous that the electrical contacting of the stimulation electrode takes place at the same time as the mechanical fixing of the electrode carrier to the electrode holder. The patient therefore does not need to take any further measures to ensure that the stimulation electrode, which is mechanically attached to the electrode holder by the electrode carrier, can be electrically contacted. The stimulation electrode arrangement is held magnetically on the electrode holder, wherein at least one of the electrode carriers establishes an electrical connection with the receptacle on the electrode holder. In particular, the electrode carrier can comprise a magnetizable metal and can be adapted to be both magnetically attached to the electrode holder and electrically conductively connected to the electrode holder.

In a refinement, the first electrode carrier can be provided with an electrical plug contact for the electrode holder, and the first end of the stimulation electrode can be electrically connected to the plug contact. The electrical plug contact can be adapted to be magnetically attached to the electrode holder. An electrical connection can thus be provided as follows: stimulation electrode connected to metal at a bottom of the funnel connected to magnet at receptacle of the electrode holder connected to stimulation source.

The stimulation electrode can be routed through at least one passage opening on the second electrode carrier and provided with a weight at its second end. The use of weights simplifies the application of a newly inserted stimulation electrode to the eye such that this can also be done by a severely visually impaired or blind patient. Because the electrode thread loaded by the weight is guided by a through-opening in the electrode carrier, the stimulation electrode is, so to speak, adaptively mounted so that it adapts easily to the contour of the eye.

Furthermore, aspects of the present disclosure relate to a set with a stimulation electrode arrangement as disclosed herein and a holding tray, wherein the first electrode carrier and the second electrode carrier are arranged in the holding tray and are adapted to be taken out of the holding tray by a magnetic force acting between an electrode holder and the first and/or second electrode carrier. The user therefore no longer has to handle a potentially delicate stimulation electrode arrangement as such, but can instead handle the holding tray with the stimulation electrode arrangement arranged therein. This further improves handling, in particular for visually impaired users. A further advantage can be that hygiene can be further improved, as the stimulation electrode arrangement does not need to be touched directly during application.

The first electrode carrier and the second electrode carrier can be mounted in the holding tray clamped with a holding force, wherein the magnetic force is greater than the holding force, such that the first and/or second electrode carrier can be taken out of the holding tray by the magnetic force. In other words, the stimulation electrode arrangement can be provided clamped in a blister pack by the first and/or second electrode carrier. The magnetic force is thereby stronger than the holding force. This prevents the electrode assembly from accidentally falling out and getting contaminated. Hygiene can thus be further improved. The holding tray is therefore designed such that the electrode carriers are held in the holding tray by a clamping force so that they cannot fall out when the holding tray is opened. The magnetic force is selected such that it is sufficient to pull the electrode carriers out of the holding tray.

The first electrode carrier and/or the second electrode carrier can be mounted in the holding tray clamped with a holding force but can be tilted (in at least one direction). One or both of the electrode carriers can be tilted slightly in the holding tray without being released from the clamping points. This allows the height difference to be compensated when the electrode holders approach. For example, a height difference between a nasal and a temporal electrode holder may arise if these are adjusted to different positions for different users. In such a case, it can happen that a nasal and a temporal electrode holder are not lowered vertically onto the electrode carriers when fixing the electrode carriers to the respective electrode holders, but are tilted slightly to compensate for a difference in height.

The holding tray can comprise a lid. In particular, the holding tray and the lid can be configured as a blister pack. This enables easy handling even for visually impaired users while at the same time improving hygiene.

Furthermore, aspects of the present disclosure relate to a device for electrostimulation of the eye, having a spectacles-like frame with a nose part, an arrangement, connected to the nose part, for holding the frame on the head of a patient and at least one electrode holder (adjustably) arranged at the frame for holding a stimulation electrode arrangement, with the stimulation electrode arrangement being the novel stimulation electrode arrangement.

Here, the nose part can have a nose clip and two eye brackets, to which respectively one electrode holder is attached.

Here, the eye brackets furthermore are preferably electrically insulated from one another and contacted individually such that a therapy with different current strengths for the two eyes is possible.

Here, the arrangement for holding the frame has two earpieces which are fixed to the nosepiece, preferably fixed thereto in foldable fashion, in a manner known per se. The arrangement for holding the frame can also comprise side arms of variable width, in particular spring-loaded variable width side arms.

In general, it is also preferable for a counter electrode to be provided for the stimulation electrode.

Furthermore, aspects of the present disclosure relate to a system for electrostimulation of the eye with a stimulation electrode arrangement according to the first aspect and a corresponding device for electrostimulation of the eye according to the second aspect. The stimulation electrode arrangement and the corresponding device can be configured as described above or as exemplarily described in the context of the present disclosure.

Furthermore, aspects of the present disclosure relate to a method for attaching a stimulation electrode arrangement to a device for electrostimulation of the eye, wherein the method comprises the following steps:
a) providing a device for electrostimulation of an eye and a stimulation electrode arrangement;
b) placing the first and/or second electrode carriers of the stimulation electrode arrangement and the electrode holder of the device at each other; and
c) magnetically attaching the first and/or second electrode carrier of the stimulation electrode arrangement to the electrode holder of the device.

In other words, in step b), the electrode holder of the device can be placed at the first and/or second electrode carrier of the stimulation electrode arrangement. Alternatively, the first and/or second electrode carrier of the stimulation electrode arrangement can be placed at the electrode holder of the device. The respective elements are thus placed close to each other, which is also a possible for a user with visual impairment, and the final attachment and orientation can be achieved by magnetic attachment.

The optional further embodiments and refinements described for the stimulation electrode arrangement according to the first aspect apply accordingly to the further aspects of the present disclosure, i.e., in particular to the set, the device, and the system for electrostimulation of the eye.

Further advantages are apparent from the description of the accompanying drawing.

It is to be understood that the features mentioned above and those to be explained below can be used not only in the respective combination specified, but also in other combinations or in isolation, without departing from the scope of the present disclosure.

### BRIEF DESCRIPTION OF THE DRAWINGS

Embodiments of aspects of the disclosure are illustrated in the drawings and are explained in more detail in the following description.

FIG. 1 shows a schematic perspective illustration of a system for electrostimulation of the eye with the novel stimulation electrode arrangement;

FIG. 2 shows a schematic illustration of the electrode holder and stimulation electrode arrangement comprising a stimulation electrode and first and second electrode carriers;

FIG. 3 shows a schematic sequence for attaching a stimulation electrode arrangement to an electrode holder;

FIG. 4 shows a first schematic perspective illustration of a stimulation electrode arrangement;

FIG. 5 shows a second schematic perspective illustration of a stimulation electrode arrangement;

FIG. 6 shows a third schematic perspective illustration of a stimulation electrode arrangement;

FIG. 7 shows a schematic cross-sectional view of a stimulation electrode arrangement which is magnetically attached to electrode holders;

FIG. 8 shows a schematic perspective illustration of an electrode holder of a device for electrostimulation of the eye;

FIG. 9 shows a schematic perspective illustration of the electrode holder of FIG. 8 with a set comprising a stimulation electrode arrangement arranged in a holding tray;

FIG. 10 shows a schematic perspective illustration of the electrode holder of FIG. 8 with a stimulation electrode arrangement of the set from FIG. 9 magnetically attached to it;

FIG. 11 shows a perspective illustration, a top view, and a sectional view of a nasal electrode carrier;

FIG. 12 shows a perspective illustration, a top view, and a sectional view of a temporal electrode carrier; and

FIG. 13 shows a flowchart of a method for attaching a stimulation electrode arrangement to a device for electrostimulation of the eye.

### DETAILED DESCRIPTION

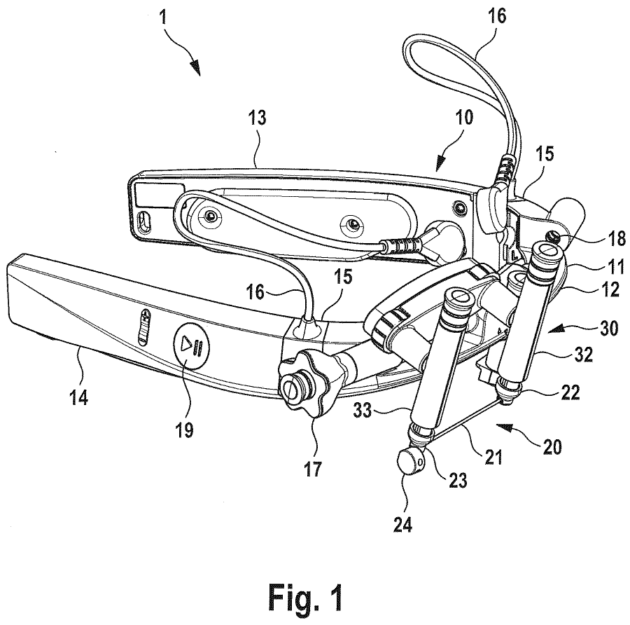

FIG. 1 shows a system 1 for electrostimulation of the eye of a user or patient. The system 1 comprises a device 10 for electrostimulation of the eye and a stimulation electrode arrangement 20.

The device 10 comprises a spectacle-like frame 11 with a nose part 12 and an arrangement connected to the nose part 12 for holding the frame on the head of the patient. The arrangement for holding the frame 11 on the head of the patient can comprise two temples or side arms 13, 14, as is usual with spectacles. Optionally, the side arms 13, 14 can be attached to the nose part 12 in a foldable manner. The side arms 13, 14 can also be connected to the nose part 12 in a width-variable way via spring elements 15. In this way, it is possible to flexibly adapt the device to the user's head.

The device further comprises an electrode holder 30 arranged at the frame 11. The electrode holder 30 can preferably be configured as a swiveling or pivotable electrode holder. In this case, the pivotable electrode holder can be moved back and forth between a raised position and a defined treatment position. In the example shown in FIG. 1, the electrode holder 30 is configured as a pivotable electrode holder and is in a raised position. This allows a patient to place the spectacle-like frame 11 with the electrode holder 30 raised on their head. This makes handling easier. In a first step, only the spectacle-like frame is put on, however it is not yet necessary to place the stimulation electrode 21 onto the eye in the same step.

For electrostimulation, the electrode holder 30 is lowered and moved into the defined treatment position so that the stimulation electrode 21 can engage with the front surface of the eye. The stimulation electrode 21 thereby curves so that it can fit snugly against the curvature of the eye surface, in particular the cornea. This is possible because the stimulation electrode 21 is flexible and can be adaptively mounted in the second electrode carrier 23 by the weight 24.

In the example shown in FIG. 1, the electrode holder can, for example, be moved back and forth between the raised position and a defined treatment position via a laterally arranged rotary handle 19. Hereby, the electrode holder 30 can be mounted on the spectacle-like frame 11 in a rotatable or pivotable manner. For example, with a bearing 18, as illustrated for an electrode holder for a left eye, which is not shown. The defined treatment position can also be referred to as the engagement position. The raised position can be referred to as the non-engagement position.

Separate electrode holders 30 can be provided for the right eye and the left eye. For simplicity, only one electrode holder 30 is illustrated in FIG. 1.

The stimulation electrode arrangement 20 is magnetically attached to the electrode holder 30. The stimulation electrode arrangement 20 comprises a first electrode carrier 22 and a second electrode carrier 23. The stimulation electrode 21 is arranged between the first electrode carrier 22 and the second electrode carrier 23. The first and/or second electrode carriers are adapted to be magnetically attached to the electrode holder 30.

In the example shown in FIG. 1, the electrode holder 30 can comprise a first, nasal electrode holder 32 and a second, temporal electrode holder 33. The first, nasal electrode holder 32 can also be referred to as the nose-side electrode holder. The second, temporal electrode holder 33 can also be referred to as the temple-side electrode holder. The first electrode carrier 22 is a nasal electrode carrier and is adapted to be magnetically attached to a receptacle of the nasal electrode holder 32. The second electrode carrier 23 is a temporal electrode carrier and is adapted to be magnetically attached to a receptacle of the temporal electrode holder 33.

In an assembled state, the stimulation electrode 21 is electrically conductively connected to a stimulation device for electrostimulation of the eye. Preferably, the electrical connection is provided in a way to be described below via the electrode holder 30. The stimulation device generates the desired electrical signals for electrostimulation. Any stimulation device can be used as the stimulation device that can emit suitable stimulation signals, in particular sine waves or pulsed electrical stimulation signals for the treatment of the eye.

The stimulation device can be integrated into the spectacle-like frame 11. This is schematically illustrated in FIG. 1 by reference sign 19 and control elements for starting or pausing the electrical stimulation. However, the stimulation device can also be provided as an external stimulation device.

In order to close the electric circuit for electrostimulation, counter electrodes 16 can also be provided, which can, for example, be attached to the patient's forehead or earlobe.

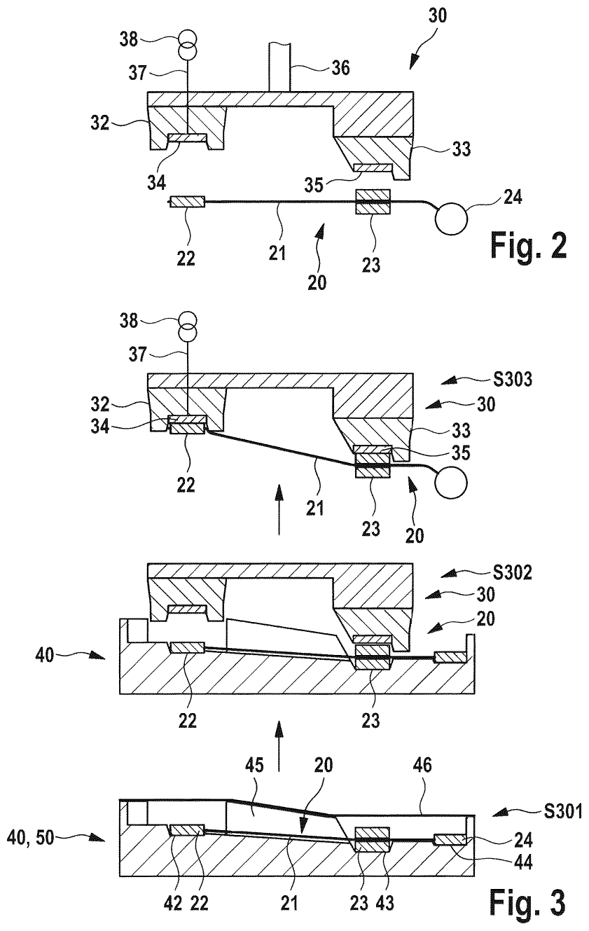

FIG. 2 shows a schematic illustration of electrode holder 30 and stimulation electrode arrangement 20. The stimulation electrode arrangement 20 comprises the first electrode carrier 22 and the second electrode carrier 23 as well as the stimulation electrode 21 arranged between them. In the example shown, the stimulation electrode 21 is attached with its first end to the first electrode carrier 22 and with its second end routed through the second electrode carrier 23 and ending in a weight 24.

By the weight 24, the stimulation electrode 21 is kept taut between the first electrode carrier 22 and the second electrode carrier 23 when attached, but due to the limited mass of the weight 24 (preferably between 1 and 20 g), it can be easily bent inwards when applied to the cornea of an eye.

The first and second electrode carriers 22, 23 and the stimulation electrode 21 are optionally combined with the weight 24 to form a stimulation electrode arrangement 20, which can be interchangeably attached to the electrode holder 30.

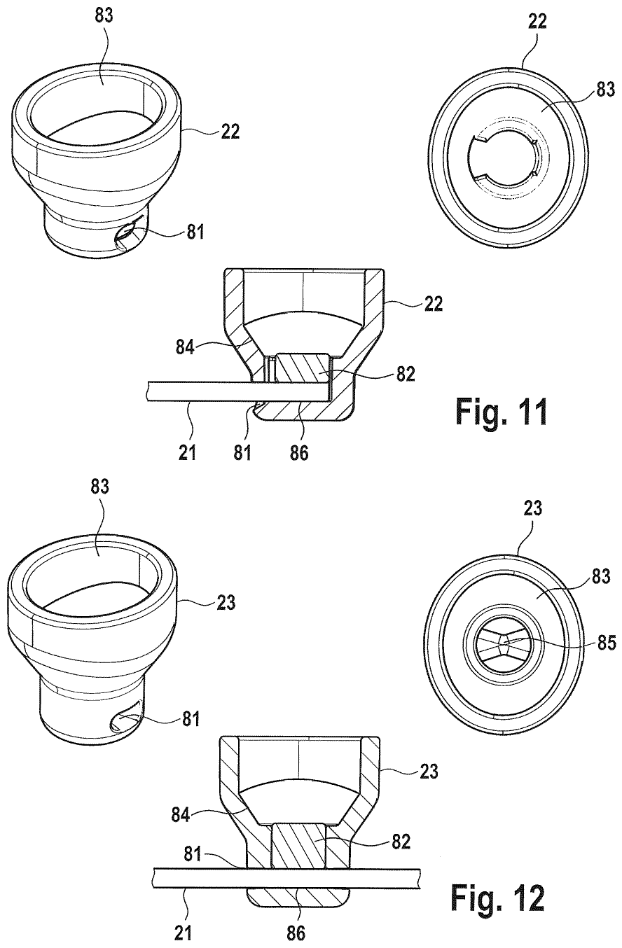

The first and/or second electrode carriers 22, 23, preferably both, are adapted to be magnetically attached to the electrode holder 30. In the example shown in FIG. 2, the electrode holder 30 comprises a first, nasal electrode holder 32 and a second, temporal electrode holder 32. The first electrode holder 32 comprises a first magnet 34, and the first electrode carrier 22 is adapted to be attached to the first electrode holder 32 by the magnet 34. Correspondingly, the second electrode holder 33 can comprise a second magnet 35, and the second electrode carrier 23 is adapted to be attached to the second electrode holder 33 by the magnet 35. For example, the first and/or second electrode carriers can comprise a magnetizable metal (see, for example, element 82 in FIG. 11, FIG. 12), which is attracted by the respective magnets of the first and second electrode holders 32, 33.

As illustrated schematically in FIG. 2, a stimulation signal for electrostimulation can be provided via the electrode holder 30 and fed to the stimulation electrode 21 of the stimulation electrode arrangement 20. An electrical connection from a stimulation device, schematically designated by reference sign 38, can be established via an electrical connection 37. In the context of the present disclosure, the stimulation device 38 may be a device for stimulating and/or measuring. For example, a stimulation signal can be generated which can be applied via the stimulation electrode arrangement 20 with the stimulation electrode 21. However, it can also, for example, be an ERG apparatus with which an electroretinogram can be recorded, which is acquired with the stimulation electrode 21 of the stimulation electrode arrangement 20.

The stimulation electrode is electrically conductively connected to the first electrode carrier 22 at least at its first end, wherein the electrode carrier 22 is adapted to be electrically conductively connected to the electrode holder 32. Preferably, the electrical connection is made electrically conductively via the magnet 34, which holds the electrode carrier 22 on the electrode holder 32. However, it is also possible that, in the alternative or in addition, an electrical connection is established via the second electrode carrier 23 and the first electrode holder 33.

It is to be understood that, in the alternative or in addition, the first electrode carrier 22 and/or the second electrode carrier 23 can respectively comprise a magnet and be adapted to be attached to the electrode holder 30 by the magnet or magnets. For cost reasons and to preserve resources, however, it is preferable if the replaceable stimulation electrode arrangement 20 does not contain a magnet. However, if a more stable attachment is required, an embodiment can provide that also the stimulation electrode arrangement 20 can comprise one or more magnets for magnetic attachment.

In FIG. 2, the electrode holder 30 can comprise a holding pin 36 with which the electrode holder can be attached to a spectacle-like frame, as for example known from DE 10 2011 055 844 B4. This enables simplified retrofitting of conventional electrostimulation systems. The proposed solution, wherein the first and/or second electrode carrier is adapted to be magnetically attached to the electrode holder 30, can be advantageously connected to a swiveling electrode holder, but is not limited thereto.

FIG. 3 shows a schematic sequence for attaching a stimulation electrode arrangement 20 to an electrode holder 30. The sequence of steps S301, S302, S303 is illustrated in FIG. 3 from bottom to top.

In a first step S301, a stimulation electrode arrangement 20 is provided with the first electrode carrier 22 and the second electrode carrier 23, as well as the stimulation electrode 21 arranged between them and the optional weight 24.

Instead of providing only the stimulation electrode arrangement 20 as such, preferably a set 50 with the stimulation electrode arrangement 20 and a holding tray 40 is provided. The first electrode carrier 22 and the second electrode carrier 23 are arranged in the holding tray 40 and are adapted such that they can be taken out of the holding tray 40 by a magnetic force acting between an electrode holder 30, 32, 33 and the first and/or second electrode carrier 22, 23. The holding tray 40 preferably comprises a cover 46. The stimulation electrode arrangement 20 can thus be provided in a protected space 45. The holding tray 40 and the cover 46 can, for example, be configured as a blister pack. The stimulation electrode arrangement 20 can thus be protected from contamination and can be easily distributed as a set in a simple way.

In the shown example, the holding tray 40 comprises a first holder 42 for the first, nasal electrode carrier 22, a second holder 43 for the second, temporal electrode carrier, and a third holder 44 for the weight 24. Thanks to the holding tray 40, the elements of the stimulation electrode arrangement 20 are provided at well-defined positions, which are adapted to the geometry of the electrode holder 30.

The holders 42, 43, 44 can be simple recesses in which the corresponding elements are received. Preferably, the first electrode carrier 22 and the second electrode carrier 23 are each mounted with a holding force in the holding tray 40. Thereby, the magnetic force between the electrode holder 30 and the first and second electrode carriers is greater than the holding force, such that the first and second electrode carriers 22, 23 can be taken out of the holding tray 40 by the magnetic force.

The taking out of the stimulation electrode arrangement 20 from the holding tray 40 is exemplarily illustrated in step S302. Here, the cover 46 has been removed from the holding tray 40. The stimulation electrode arrangement is now accessible from the top side. The electrode holder 30 can be lowered onto the stimulation electrode arrangement 20, which is still located in the holding tray 40, and receive it magnetically.

This can further simplify handling, in particular for visually impaired persons. The holding tray 40 is a geometrically larger element than the rather fragile stimulation electrode arrangement 20. A further advantage may be that, thanks to the magnetic attachment, pressing the stimulation electrode arrangement 20 into the electrode holder 30 is not necessary. Such pressing can alter the adjustment of the electrode holder and require readjustment by a skilled person.

The holding tray 40 can be configured such that the first electrode carrier 22 is arranged in the holding tray at a higher level than the second electrode carrier 23. This can facilitate handling, as a nasal electrode holder 32 and a temporal electrode holder 33 often have different distances. The different distances are illustrated as different levels in the sectional view in FIG. 3. Alternatively or in addition, the first electrode carrier 22 and/or the second electrode carrier 23 are mounted in the holding tray 40 clamped with a clamping force but can be tilted in at least one direction. This allows height differences or different eye distances when adjusting the nasal and temporal electrode holders 32, 33 to be compensated for. Furthermore, imprecise placement of the set 50 with the holding tray 40 relative to the electrode holder 30 can be compensated for.

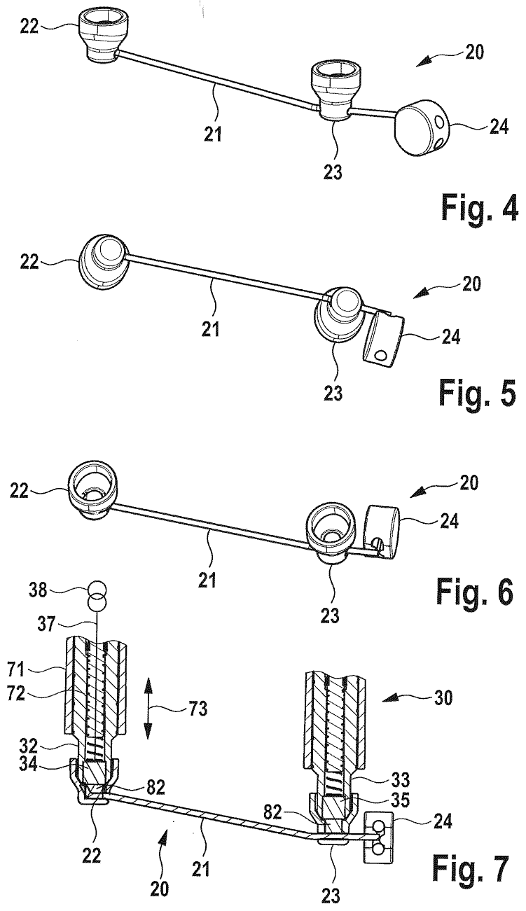

FIG. 4 to FIG. 6 illustrate schematic perspective views of a stimulation electrode arrangement 20 which is adapted to be magnetically attached to an electrode holder 30.

FIG. 7 shows a sectional view, wherein the stimulation electrode arrangement 20 is attached to an electrode holder 30 comprising a nasal first electrode holder 32 and a temporal second electrode holder 33. The stimulation electrode arrangement 20 comprises the first electrode carrier 22 and the second electrode carrier 23, wherein the stimulation electrode 21 is arranged between the first electrode carrier 22 and the second electrode carrier 23. Furthermore, the stimulation electrode arrangement comprises the optional weight 24 as described above.

The first electrode carrier 22, in the present example the nasal electrode carrier, is illustrated in FIG. 11 with a perspective view (top left in FIG. 11), a top view (top right in FIG. 11), and a sectional view (bottom in FIG. 11).

The second electrode carrier 32, in the present example the nasal electrode carrier, is illustrated in FIG. 12 with a perspective view (top left in FIG. 12), a top view (top right in FIG. 12) and a sectional view (bottom in FIG. 12).

In order to further facilitate the attachment of the stimulation electrode arrangement 20, the first and/or second electrode carriers 22, 23 are adapted to be attached self-aligning at the electrode holder 30.

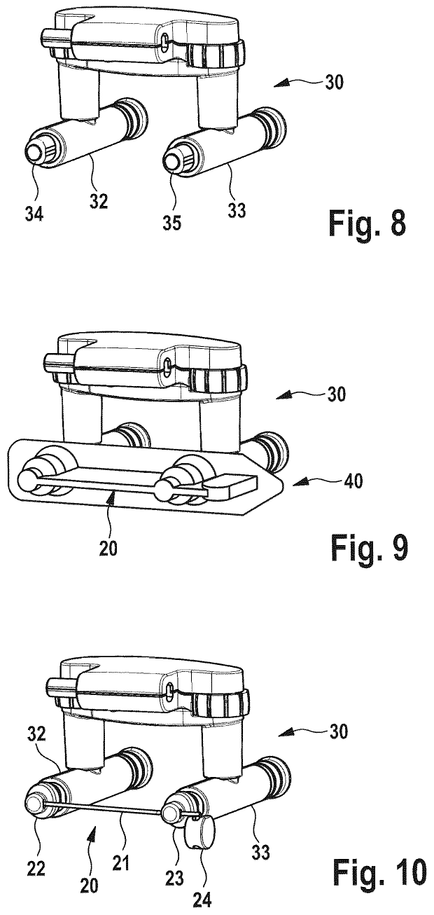

As shown in the cross-sectional views in FIG. 11 and FIG. 12, the first, nasal, and second, temporal, electrode carriers can each comprise a funnel 84. The electrode holder 30 can, as for example illustrated in FIG. 7 and FIG. 8, have a corresponding nasal electrode holder 32 and temporal electrode holder 33. The funnels 84 of the nasal and temporal electrode carriers 22, 23 can be placed on the corresponding pin-shaped receptacles of the nasal and temporal electrode holders 32, 33.

As shown in the top views in FIG. 11 and FIG. 12, the first and second electrode carriers 22, 23 comprise an oval receptacle 83 for the first and second electrode holders. The receptacle 83 is longer than it is wide, i.e., oval within the context of the present disclosure. Accordingly, the first and second electrode holders 32, 33, as for example shown in FIG. 8, comprise corresponding receptacle pins with an oval shape. However, other embodiments are also possible, wherein the first and/or second electrode carriers 22, 23 are adapted to be attached to the electrode holders 30, 32, 33 in a defined orientation.

As shown in FIG. 11 and FIG. 12, the first and second electrode carriers comprise an opening 81 into which the stimulation electrode 21 is inserted. As shown in the lower illustration in FIG. 12, the second, temporal electrode carrier 23 can be a through opening 81 through which the stimulation electrode 21 coming from the first, nasal electrode carrier 22 is routed to the weight 24. As shown in the top view in FIG. 12, a taper 85 can be provided in the through opening. The taper 85 can be adapted to improve or, if necessary, inhibit the routing of the stimulation electrode 21 by the through-opening. In the example shown in FIG. 11, it is however not necessary to pass the stimulation electrode 21 completely through the electrode carrier 22.

In the shown embodiment, a magnetizable metal element 82 at the bottom 86 of the funnel 84 is adapted to provide both an electrical contact to the stimulation electrode 21 and to magnetically attach it to the electrode holder. It is to be understood that the electrode carriers 22, 23 can also comprise magnets, in particular electrically conductive magnets, instead of magnetizable metal elements 82.

FIG. 7 shows a schematic cross-sectional view of a stimulation electrode arrangement 20 which is magnetically attached to the electrode holder 30. Here, the first electrode carrier 22 is magnetically attached to the first electrode holder 32. The second electrode carrier 23 is magnetically attached to the second electrode holder 33. The first electrode carrier 22 is configured as illustrated in FIG. 11. The second electrode carrier 23 is configured as illustrated in FIG. 12. The first electrode holder 32 comprises a first magnet 34, which attracts the magnetizable metal element 82 at the bottom 86 of the funnel 84 (see FIG. 11) of the first electrode carrier 22 and attaches the first electrode carrier 22 by magnetic force. The second electrode holder 33 comprises a second magnet 35, which attracts the magnetizable metal element 82 at the bottom 86 of the funnel 84 (see FIG. 12) of the second electrode holder 22 and attaches the electrode holder 22 by magnetic force.

An electrical connection between the stimulation electrode 21 and the stimulation device 38 is also schematically illustrated in FIG. 7. Electrical signal transmission to the stimulation electrode 21 takes place via a cable 37, the first magnet 34 of the first electrode holder 32, and the magnetizable metal element 82 of the first electrode carrier 22. It shall be understood that the cable is only schematically illustrated and that another electrical connection, for example via conductive elements of the first electrode holder 32, can also be provided.

The first and/or second electrode holder 32, 33 can be configured to be length-adjustable. For example, an inner part 72 can be moved relative to an outer sleeve 71. This allows the distance between the electrode holder 32, 33 and the eye of the user to be adjusted. However, the distance between the first and second electrode holders 32, 33 is preferably fixed. The fixed distance enables the stimulation electrode arrangement to be placed in a holding tray at a defined distance and to be taken out of the tray. The first and second electrode carriers are thereby preferably arranged at the same distance from each other as the distance between the first and second electrode holders 32, 33.

FIG. 8 to FIG. 10 illustrate the process of attaching a stimulation electrode arrangement 20 to an electrode holder 30 in perspective views.

Thereby, FIG. 8 illustrates a schematic perspective view of the electrode holder 30 with a nasal first electrode holder 32 and a temporal second electrode holder 33. The nasal electrode holder 32 comprises a pin-shaped receptacle with a first magnet 34. The temporal electrode holder 33 comprises a pin-shaped receptacle with a second magnet 35. The receptacles are each configured oval. This allows the electrode carriers 22, 23 to be attached to the electrode holders 32, 33 in a defined orientation.

As shown in FIG. 9, in the next step, the set 50 with the receptacle 40 and the stimulation electrode arrangement 20 arranged therein can be placed at the electrode holder 30 such that the stimulation electrode arrangement 20 can be taken out of the receptacle 40 by magnetic force. Hence, the user does not have to touch the stimulation electrode directly. This makes handling easier for a visually impaired user. In addition, hygiene during handling is improved.

FIG. 10 shows a schematic perspective illustration of the electrode holder 30 with a stimulation electrode arrangement 20 magnetically attached to it.

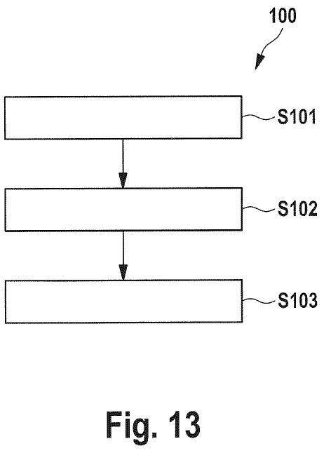

FIG. 13 shows a flowchart of a method 100 for attaching a stimulation electrode arrangement to a device for electrostimulation of the eye.

In a first step S101, a device for electrostimulation of an eye with an electrode holder 30, as disclosed herein, and a stimulation electrode arrangement, as disclosed herein, are provided.

In a second step S102, the first and/or second electrode carriers of the stimulation electrode arrangement and the electrode holder of the device are placed or arranged at each other.

In a third step S103, the first and/or second electrode carriers of the stimulation electrode arrangement are magnetically attached to the electrode holder of the device.

In conclusion, according to aspects of the present disclosure, the handling of a stimulation electrode for electrostimulation of the eye, in particular for visually impaired users, can be further improved.

It is to be understood that the foregoing description is of one or more embodiments of the present disclosure. The present disclosure is not limited to the particular embodiment(s) disclosed herein, but rather is defined solely by the claims below. Furthermore, the statements contained in the foregoing description relate to the disclosed embodiment(s) and are not to be construed as limitations on the scope of the disclosure or on the definition of terms used in the claims, except where a term or phrase is expressly defined above. Various other embodiments and various changes and modifications to the disclosed embodiment(s) will become apparent to those skilled in the art.

As used in this specification and claims, the terms “e.g.,” “for example,” “for instance,” “such as,” and “like,” and the verbs “comprising,” “having,” “including,” and their other verb forms, when used in conjunction with a listing of one or more components or other items, are each to be construed as open-ended, meaning that the listing is not to be considered as excluding other, additional components or items. Other terms are to be construed using their broadest reasonable meaning unless they are used in a context that requires a different interpretation. In addition, the term “and/or” is to be construed as an inclusive OR. Therefore, for example, the phrase “A, B, and/or C” is to be interpreted as covering all of the following: “A”; “B”; “C”; “A and B”; “A and C”; “B and C”; and “A, B, and C.”

1-22. (canceled)

23. A stimulation electrode arrangement for a device for electrostimulation of the eye, wherein the device comprises a spectacle-shaped frame with a nose part, an arrangement connected to the nose part for holding the frame on a head of a patient, and at least one electrode holder arranged at the frame, the stimulation electrode arrangement comprising:
a first electrode carrier;
a second electrode carrier; and
a stimulation electrode configured to be attached to said electrode holder, wherein the stimulation electrode is arranged between the first electrode carrier and the second electrode carrier, and wherein at least one of the first electrode carrier and the second electrode carrier is adapted to be magnetically attached to said electrode holder.

24. The stimulation electrode arrangement of claim 23, wherein the first electrode carrier is adapted to be magnetically attached to a first electrode holder, and wherein the second electrode carrier is adapted to be magnetically attached to a second electrode holder.

25. The stimulation electrode arrangement of claim 23, wherein the first electrode carrier is a nasal electrode carrier and is adapted to be magnetically attached to a nasal receptacle of said electrode holder, and wherein the second electrode carrier is a temporal electrode carrier and is adapted to be magnetically attached to a temporal receptacle of said electrode holder.

26. The stimulation electrode arrangement of claim 23, wherein at least one of the first electrode carrier and the second electrode carrier comprises a magnet, and wherein the at least one first and second electrode carrier is adapted to be attached to said electrode holder by the magnet.

27. The stimulation electrode arrangement of claim 23, wherein said at least one electrode holder comprises a magnet, and wherein at least one of the first electrode carrier and the second electrode carrier is adapted to be attached to said electrode holder by the magnet.

28. The stimulation electrode arrangement of claim 27, wherein at least one of the first electrode carrier and the second electrode carrier comprises a magnetizable metal, and wherein the magnetizable metal is adapted to be attached to said electrode holder by the magnet.

29. The stimulation electrode arrangement of claim 23, wherein at least one the first electrode holder and the second electrode carrier is configured to be self-aligning when attached at said electrode holder.

30. The stimulation electrode arrangement of claim 29, wherein at least one the first electrode holder and the second electrode carrier comprises a funnel, and wherein the at least one first and second electrode holder is configured to be self-aligning at said electrode holder by the funnel.

31. The stimulation electrode arrangement of claim 30, further comprising:
an electrical contact arranged at a bottom of the funnel, wherein the electrical contact is electrically conductively connected to the stimulation electrode.

32. The stimulation electrode arrangement of claim 31, further comprising:
a magnetizable metal element arranged at the bottom of the funnel, wherein the magnetizable metal element is adapted to provide both the electrical contact to the stimulation electrode and to be magnetically attached to said electrode holder.

33. The stimulation electrode arrangement of claim 29, wherein at least one of the first electrode carrier and the second electrode carrier is adapted to be attached in a defined orientation at said electrode holder, and wherein said at least one electrode holder comprises an oval receptacle for at least one of the first electrode carrier and the second electrode carrier.

34. The stimulation electrode arrangement of claim 23, wherein the stimulation electrode is a contact lens provided with at least one wire-shaped connection and electrode structures.

35. The stimulation electrode arrangement of claim 23, wherein at least a first end of the stimulation electrode is electrically conductively connected to the first electrode carrier, and wherein the first electrode carrier is adapted to be electrically conductively connected to said electrode holder.

36. The stimulation electrode arrangement of claim 23, wherein the first electrode carrier and the second electrode carrier are arranged in a holding tray and are adapted to be taken out of the holding tray by a magnetic force acting between said electrode holder and one of the first electrode carrier and the second electrode carrier.

37. The stimulation electrode arrangement of claim 36, wherein the first electrode carrier and the second electrode carrier are mounted in the holding tray and clamped with a holding force, and wherein the magnetic force is greater than the holding force, such that the first electrode carrier and the second electrode carrier can be removed from the holding tray by the magnetic force.

38. The stimulation electrode arrangement of claim 37, wherein the first electrode carrier and the second electrode carrier are mounted in the holding tray with the holding force but can be tilted in at least one direction.

39. The stimulation electrode arrangement of claim 36, wherein the holding tray comprises a cover, and wherein the holding tray and the cover are configured as a blister pack.

40. A system for electrostimulation of an eye, comprising:
a device for electrostimulation of said eye, the device comprising:
a spectacle-shaped frame with a nose part;
an arrangement connected to the nose part for holding the spectacle-shaped frame on a head of a user; and
at least one electrode holder arranged at the spectacle-shaped frame for a stimulation electrode arrangement; and
a stimulation electrode configured to be attached to the at least one electrode holder, the stimulation electrode comprising:
a first electrode carrier; and
a second electrode carrier, wherein the stimulation electrode is arranged between the first electrode carrier and the second electrode carrier, and wherein the at least one electrode holder is adapted to magnetically attach at lease one of the first and the second electrode carriers at the at least one electrode holder.

41. A method for attaching a stimulation electrode arrangement to a device for electrostimulation of an eye, the method comprising steps of:
providing said device for electrostimulation of said eye, wherein said device includes:
a spectacle-shaped frame with a nose part;
an arrangement connected to the nose part for holding the spectacle-shaped frame on a head of a user; and
at least one electrode holder arranged at the spectacle-shaped frame;
providing said stimulation electrode arrangement, wherein said stimulation electrode arrangement includes:
a first electrode carrier;
a second electrode carrier; and
a stimulation electrode configured to be attached to the at least one electrode holder, wherein the stimulation electrode is arranged between the first electrode carrier and the second electrode carrier, and wherein at least one of the first electrode carrier and the second electrode carrier is adapted to be magnetically attached to the electrode holder;
placing the first and second electrode carriers of said stimulation electrode arrangement and the electrode holder of said device at each other; and
magnetically attaching the first and second electrode carriers of said stimulation electrode arrangement to the electrode holder of said device.
